(19) United States
(12) Patent Application Publication
(10) Pub. No.: US 2015/0094647 A1
(43) Pub. Date: Apr. 2, 2015

(54) METHODS AND APPARATUS FOR DELIVERY OF MOLECULES ACROSS LAYERS OF TISSUE

(71) Applicants: Sameer Kalghatgi, Copley, OH (US); Daphne Pappas Antonakas, Hudson, OH (US); Tsung-Chan Tsai, Cuyahoga Falls, OH (US); Robert L. Gray, Hudson, OH (US)

(72) Inventors: Sameer Kalghatgi, Copley, OH (US); Daphne Pappas Antonakas, Hudson, OH (US); Tsung-Chan Tsai, Cuyahoga Falls, OH (US); Robert L. Gray, Hudson, OH (US)

(73) Assignee: EP Technologies LLC, Akron, OH (US)

(21) Appl. No.: 14/500,144

(22) Filed: Sep. 29, 2014

Related U.S. Application Data
(60) Provisional application No. 61/883,701, filed on Sep. 27, 2013.

(51) Int. Cl. A61M 37/00; H05H 1/24; A61N 1/44

(52) U.S. Cl. CPC A61M 37/00; A61N 1/44; H05H 1/2406; A61M 2037/0007; B82Y 5/00
USPC 604/23; 977/774; 977/906

(57) ABSTRACT

Exemplary methods of opening pores and moving molecules into tissue comprising, applying plasma to the surface of tissue and applying a carrier including one or more molecules to the surface of the tissue are disclosed herein.

### RELATED APPLICATIONS

This non-provisional utility patent application claims priority to and the benefits of U.S. Provisional Patent Application Ser. No. 61/883,701 filed on Sep. 27, 2013 and entitled METHODS AND APPARATUS FOR DELIVERY OF MOLECULES ACROSS LAYERS OF THE SKIN. This application is incorporated herein by reference in its entirety.

### TECHNICAL FIELD

The present invention relates generally to methods and solutions for enabling or enhancing intracellular or intercellular transportation of molecules across tissue including layers of the skin using non-thermal plasma, and more particularly for opening pores in skin or tissue and transporting one or more molecules across layers of skin or tissue for deep tissue sanitization; delivery of vaccines, drugs and cosmetics; improvement of skin health; and the like.

### BACKGROUND OF THE INVENTION

Transdermal delivery is localized, non-invasive, and has the potential for sustained and controlled release of drugs, and other molecules. In addition, transdermal drug delivery avoids first-pass metabolism, which reduces the concentration of the drug before the drug reaches the circulatory system. In addition, percutaneous absorption minimizes the risk of irritation of gastrointestinal tract, minimizes pain and other complications associated with parenteral administration.

Transdermal delivery, however, requires molecules to pass through the skin. FIG. 1 illustrates the layers of the skin 100. The outer layer of the skin 100 is the stratum corneum (“SC”) 102. The SC 102 is composed of dead, flattened, keratin-rich cells, called corneocytes. These dense cells are surrounded by a complex mixture of intercellular lipids—namely, ceramides, free fatty acids, cholesterol and cholesterol sulfate. The predominant diffusional path for a molecule crossing the SC appears to be intercellular. The remaining layers of the skin are the epidermis (viable epidermis) 104, the dermis 106, and the subcutaneous tissue 108.

Only a small percentage of compounds can be delivered transdermally because skin 100 has barrier properties, namely the highly lipophilic SC 102, that prevents molecules from penetrating the skin. As a result, only, molecules with a molecular weight (MW) of less than 500 Dalton can be administered topically or percutaneously. Often, for pharmaceutical applications, the development of innovative compounds is restricted to a MW of less than 500 Dalton when topical dermatological therapy, percutaneous systemic therapy or vaccination is the objective. In addition, transport of most drugs across the skin is very slow, and lag times to reach steady-state fluxes are measured in hours. Achievement of a therapeutically effective drug level is therefore difficult without artificially enhancing skin permeation.

A number of chemical and physical enhancement techniques have been developed in an attempt to compromise the skin barrier function in a reversible manner. These attempts may be classified as passive and active methods.

Passive methods for enhancing transdermal drug delivery include the use of vehicles such as ointments, creams, gels and passive patch technology. In addition, there are other passive methods that artificially damage the barrier in order to allow improved permeation of active substances, such as, for example, micro-needles that produce small holes having a depth of approximately 100-200 μm in the skin to allow improved permeation. The amount of substance that can be delivered using these methods is limited because the barrier properties of the skin are not fundamentally changed.

Active methods for enhancing transdermal drug delivery systems involve the use of external energy to act as a driving force and/or act to reduce the SC barrier resistance and enhance permeation of drug molecules into the skin. Iontophoresis and electroporation are two common methods of active transdermal drug delivery systems.

Iontophoresis is the process of increasing the permeation of electrically charged drugs into skin by the application of an electric current. The amount of a compound delivered is directly proportional to the quantity of charge passed; i.e. it depends on the applied current, the duration of current application and the surface area of the skin in contact with the active electrode compartment. Advantages of iontophoresis include an improved onset time and also a more rapid offset time—that is, once the current is switched off, there is no further transportation of the compound.

To deliver drugs using iontophoresis, a drug is applied under an electrode of the same charge as the drug and return electrode having an opposite charge is placed on the body surface. A current below the level of the patient's pain threshold is applied for an appropriate length of time. Because like charges repel one another, the electrical current increases the permeation of the drug into surface tissues, without altering the structure of the SC. Iontophoresis transports drugs primarily through existing pathways in skin, such as hair follicles and sweat glands. Iontophoresis is typically used when a low level delivery is desired over a long time period. Iontophoresis involves the use of relatively low transdermal voltages (<100 V).

Transdermal absorption of drugs through iontophoresis is affected by drug concentration, polarity of drugs, pH of donor solution, ionic competition, ionic strength, electrode polarity, etc. Iontophoresis has safety concerns due to the use of electrical contacts on the skin, which may result in patient discomfort, muscle contraction, pain and sometimes-even skin damage and burns.

Electroporation is a method for transdermal drug delivery that consists of applying high-voltage pulses to skin. The applied high-voltage plays a dual role. First, it creates new pathways for enhancing drug permeability and second, it provides an electrical force for driving like charged molecules through the newly created pores. Electroporation is usually used on the unilamellar phospholipid bilayers of cell membranes. However, it has been demonstrated that electroporation of skin is feasible, even though the SC contains multilamellar, intercellular lipid bilayers with phospholipids and no living cells.

Electroporation of skin requires high transdermal voltages (˜100 V or more, usually >100 V). In transdermal electroporation, the predominant voltage drop of an applied electric pulse to the skin develops across the SC. This voltage distribution causes electric breakdown (electroporation) of the SC. If the voltage of the applied pulses exceeds a voltage threshold of about 75 to 100 V, micro channels or “local transport regions” are created through the breakdown sites of the SC.

DNA introduction is the most common use for electroporation. Electroporation of isolated cells has also been used for (1) introduction of enzymes, antibodies, and other biochemical reagents for intracellular assays; (2) selective biochemical loading of one size cell in the presence of many smaller cells; (3) introduction of virus and other particles; (4) cell killing under nontoxic conditions; and (5) insertion of membrane macromolecules into the cell membrane.

The presence of electrodes in contact with skin/tissue and the delivery of current into skin/tissue in this manner leads to patient discomfort, muscle contractions, pain and sometimes even skin damage and burns. In addition, electroporation often takes hours, e.g. 6 to 24 hours, to drive therapeutic amount of drugs or other molecules transdermally.

U.S. Pat. No. 8,455,228, entitled “Method to Facilitate Directed Delivery and Electroporation Using a Charged Steam”, state that “the method and apparatus in accordance with the present invention are effective in using an electrical field to adjust the electrochemical potential of a target molecule thereby providing molecular transport of the target molecule into and/or across the tissue by a diffusive transport mechanism.” The '228 patent discloses a first embodiment with dielectric properties to assure that it will hold a charge sufficient to polarize charged entities contained within a vessel and a plurality of electroporation applicators. The '228 patent disclosure suffers from several deficiencies. First, it requires molecules that may be polarized or charged, second it requires electroporation applicators and third, the molecule is contacted with plasma during the process, which may modify the molecular structure causing adverse results.

The '228 patent also discloses a second embodiment utilizing a plasma jet with a ground ring around an inner chamber. The disclosure related to this device containing cells suspended in fluid in the inner chamber and promoting uptake into the cells; or injecting plasmid intradermally and exposure of the injection site to plasma.

US patent publication No. 2014/0188071 discloses a method of applying a substance to the skin and applying plasma to the same area. The '071 publication disclose an open cell foam to hold a drugs, water etc. and applies plasma through the open cell foam. Applying plasma through the open cell foam and contacting the drugs with plasma may alter the molecular structure of the drugs and cause undesirable side effects and/or render the drug ineffective.

US patent publication 2012/0288934 discloses a plasma jet and the active substance is applied to the skin with the gas stream of the plasma jet and is transported onto the region of the living cells through the barrier door that has been opened by the plasma. Applying the active substance with the gas stream of the plasma jet may alter the molecular structure of the active substance and cause undesirable side effects and/or render the active substance ineffective.

### SUMMARY

Methods of delivering or moving molecules into skin comprising, applying plasma to the surface of skin to open pores in skin; applying a carrier having one or more molecules having a molecular weight of greater than 500 Da to the surface of the skin; and transporting the molecules through the pores to the desired depth are disclosed herein.

Additionally methods for enhancing permeation by applying plasma to the surface of skin to open pores in skin; then applying a carrier having one or more molecules having a molecular weight of less than 500 Da to the surface of skin; and transporting the molecules through the pores to a desired depth are disclosed herein.

Additionally methods for delivering molecules through the skin by applying plasma to the surface of skin to open pores in skin; applying a carrier having one or more molecules having a molecular weight greater than 500 Da to the surface of the skin for a predetermined amount of time; and then applying plasma again.

Exemplary methods of applying sanitizer to skin are disclosed herein. An exemplary method includes applying plasma to the surface of skin to open reversible pores in skin; then applying sanitizer to the surface of the skin; and transporting the sanitizer through the pores to the desired depth.

Exemplary methods of transdermal drug delivery are disclosed herein. An exemplary method includes applying plasma to the surface of skin to open pores in skin; then applying drugs to the surface of skin; and transporting the drugs through the pores to the desired depth.

Exemplary methods of transdermal vaccination are disclosed herein. An exemplary method includes applying plasma to the surface of skin to open pores in skin; then applying vaccines to the surface of skin; and transporting the vaccines through the pores to the desired depth.

Exemplary methods of treating acne are disclosed herein. An exemplary method includes treating one or more sites of acne on skin with plasma and then applying an antimicrobial to the one or more sites of acne.

Exemplary methods of applying moisturizer to skin are disclosed herein. An exemplary method includes applying plasma to the surface of skin to open pores in skin; then applying a moisturizer to the surface of the skin; and transporting the moisturizer through the pores to a desired depth.

Exemplary methods of applying cosmetics to skin are disclosed herein. An exemplary method includes applying plasma to the surface of skin to open pores in skin; then applying cosmetics to the surface of the skin; and transporting the moisturizer through the pores to the desired depth.

### BRIEF DESCRIPTION OF THE DRAWINGS

These and other features and advantages of the present invention will become better understood with regard to the following description and accompanying drawings in which:

FIG. 1 is an exemplary illustration of the layers of skin;

FIG. 2 illustrates an exemplary transdermal delivery system for opening pores in the skin and delivering or moving molecules through skin;

FIG. 3 illustrates another exemplary transdermal delivery system for opening pores in the skin and delivering or moving molecules across the skin

FIG. 4 illustrated a third exemplary transdermal delivery system for opening pores in the skin and delivering or moving molecules across the skin;

FIG. 5 is yet another exemplary transdermal delivery system for opening pores in the skin and delivering or moving molecules across the skin;

FIG. 6 is a plan view of the electrodes of FIG. 5;

FIG. 7 is a chart comparing a selected parameter to depth of permeation;

FIG. 8 is another chart comparing selected parameters to depth of permeation; and

FIG. 9 is another chart comparing selected parameters to depth of permeation.

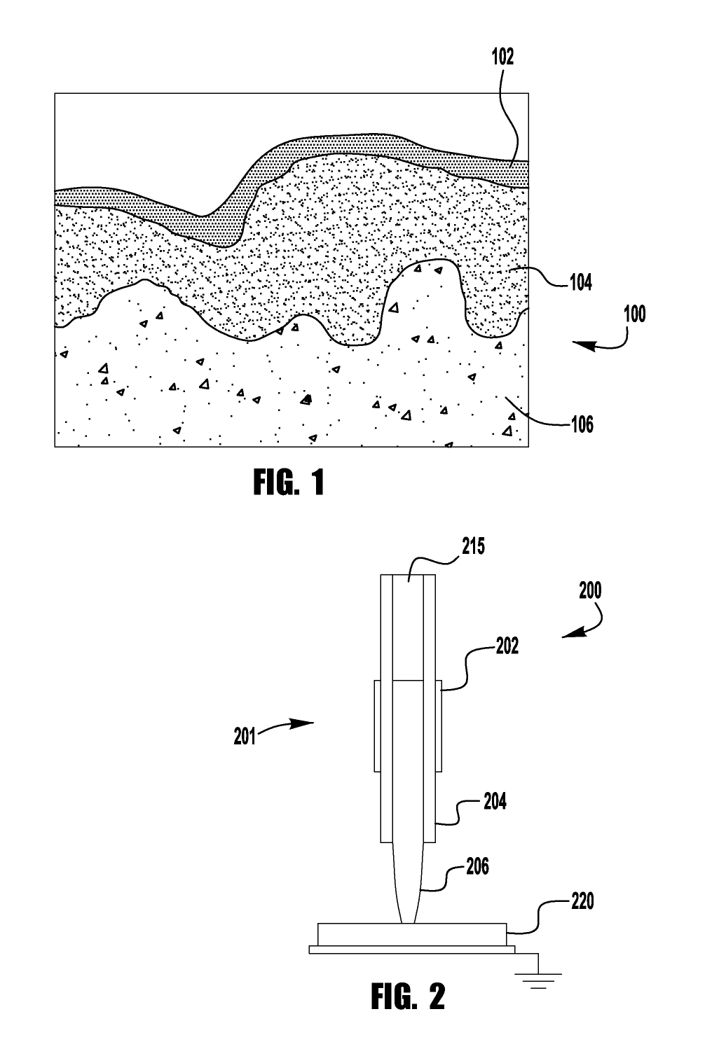

### DESCRIPTION

FIG. 2 illustrates an exemplary embodiment of a transdermal delivery system 200 for opening pores in the skin 220 and delivering or moving molecules through the open pores in the skin 220. The exemplary transdermal delivery system 200 includes a non-thermal plasma generator 201 that includes a high voltage tubular electrode 202 and a borosilicate glass tube 204. Plasma generator 201 is a floating-electrode dielectric barrier discharge (DBD) plasma generator that generates a plasma “jet” 206.

Plasma generator 201 includes a gas feed 215. Exemplary gases that may be used to feed the plasma jet include He, He+O₂, N₂, He+N₂, Ar, Ar+O₂, Ar+N₂, and the like. Gases resulting from the evaporation of liquid solutions can also be used. Examples of vaporized liquids may include water, ethanol, organic solvents and the like. These vaporized liquids may be mixed with additive compounds. The evaporated liquids and additives may be used with the gases identified above in various concentrations or without the gases. Plasma generator 201 includes a power supply, not shown. The power supply is a high voltage supply and may have a number of different wave forms, such as, for example, a constant, ramp-up, ramp-down, pulsed, nanosecond pulsed, microsecond pulsed, square, sinusoidal, random, in-phase, out-of-phase, and the like. In some of the exemplary embodiments, the power supply was a microsecond pulsed power supply. The plasma 206 was generated by applying alternating polarity pulsed voltage. The voltage had a pulse width of between about 1-10 μs at an operating frequency of 50 Hz to 3.5 kHz with a rise time of 5V/ns and a magnitude of about ˜20 kV (peak-to-peak) at a power density of 0.1-10 W/cm². During operation, the plasma jet 206 is in direct contact with the skin 220.

The plasma allows the electric field to reach the skin and deposit electrical charges to develop a voltage potential across the skin, which leads to intracellular and intercellular poration. In an exemplary system disclosed herein, the working gas of the plasma jet 206 was helium with a flow rate at 3 slm (standard liters per minute); the operating frequency was 3500 Hz, at a pulse width of 1 μs and a duty cycle of 100%. The spacing between the jet nozzle and the skin to be treated was kept at 5 mm. The use of helium gas reduced the plasma temperature and compared to air, increased the working distance to the skin 220. Plasmaporation, described above is non-invasive as the plasma electrode is not in contact with the tissue or substrate to be treated.

With respect to intracellular poration, the transmembrane voltage of fluid lipid bilayer membranes reaches at least about 0.2 V. The transmembrane voltage charges the lipid bilayer membranes, causes rapid, localized structural rearrangements within the membrane and causes transitions to water-filled membrane structures, which perforate the membrane forming “aqueous pathways” or “pores.” The aqueous pathways or pores allow an overall increase in ionic and molecular transport. The transmembrane voltage is believed to create primary membrane “pores” with a minimum radius of about approximately 1 nm. In addition, the applied electric field results in rapid polarization changes that deform mechanically unconstrained cell membranes (e.g., suspended vesicles and cells) and cause ionic charge redistribution governed by electrolyte conductivities.

The electrical pulses used to generate the plasma jet 206 also cause intercellular poration. The SC, which is about 15-25 μm thick, is the most electrically resistive part of skin. The application of electrical pulses used to generate the plasma jet 206 gives rise to a transdermal voltage ranging between about 50V and about 100V, which causes poration of the multilamellar bilayers within the SC. At these levels of applied transdermal voltage, poration of cell linings of sweat ducts and hair follicles also occurs.

Upon removal of the plasma source from the treated area the pores tend to close again and thus, the process is reversible. Some pores remain open for an extended period of time, during which molecules can continue to cross the cell membrane via diffusion. It has been discovered that in some embodiments, the pores remain open for less than about 5 minutes. Experimental results demonstrated that a 10 kDa Dextran molecule applied to a plasma treated area was transported through open pores in the SC when applied within 0 to about 5 minutes. After 5 minutes, the 10 kDa Dextran molecules no longer passed through the SC.

When electric pulses are applied to the skin, the absorbed energy can cause localized heating and damage to the skin. Energy greater than 50 J/cm² deposited on intact skin results in second degree burns and thermal damage to the underlying intact skin. One method of overcoming this problem is to apply short duration pulses repetitively, which allows the same amount of energy that would otherwise cause damage to be transferred without causing localized heating and skin damage. In some embodiments, the energy deposited on intact skin is less than about 25 J/cm², in some embodiments, the energy deposited on intact skin is less than about 10 J/cm², in some embodiments, the energy deposited on intact skin is less than about 5 J/cm², and in some embodiments, the energy deposited on intact skin is less than about 3 J/cm². However, when treating wounds, the energy may be increased, to for example, 500 J/cm², without causing burns. In some embodiments, energy in the range of 500 J/cm² may be used to coagulate blood.

In addition, damage to the skin may occur from localized plasma micro-discharges, also known as “streamers,” that occur with non-uniform electric fields. This problem may be overcome by creating a uniform electric field. In some embodiments, helium gas may be used as the gas supplied to plasma generator 201. It has been discovered, that use of helium provides a uniform plasma field and minimizes streamers. In addition, a nanosecond pulsed power supply provides a more uniform plasma field and accordingly, less pain and/or potential damage to skin. Also, skin damage can be avoided by reducing the power level, frequency, duty-cycle and pulse duration of the power supply and by increasing the spacing between the plasma electrode and skin to be treated.

After the application of plasma to cause plasmaporation and once the plasma generating device 206 is turned off, the multilamellar system of aqueous pathways remain open for a period of time that may be up to about a few minutes to few hours.

Other types of plasma generators may be used for transdermal delivery systems, such as, for example, nanosecond pulsed DBD plasma, microsecond pulsed DBD plasma, sinusoidal DBD plasma, resistive barrier discharge plasma, surface DBD plasma, 2-D or 3-D array of DBD plasma jets operating under a continuous mode or under a controlled duty cycle ranging from 1-100% and the like. It is important to note that not all plasma generators may be used to successfully induce poration. Thermal plasmas, gliding arc discharges, DC hollow cathode discharge, positive or negative corona generators and plasmatron generators are examples of plasma generators that are not suitable for use in plasmaporation. Such plasma generators either deliver conduction current, which causes thermal damage, muscle contraction and pain or do not deliver sufficient charges to the substrate being treated, which would mean no or very weak applied electric field and hence no induced poration.

Suitable plasma generators have dominating currents that are displacement currents at low power and/or high frequencies. Displacement current has units of electric current density, and an associated magnetic field just as conduction current has, however, it is not an electric current of moving charges, but rather a time-varying electric field. The electric field is applied to the skin by an insulated electrode that is not in contact with the skin. Because the electrode is insulated and is not in contact with the skin, there is no flow of conduction current into the skin, which would cause thermal damage, muscle contraction and pain that is associated with electroporation.

For larger treatment areas, electrode configurations consisting of multiple plasma jets or larger area flat electrodes (not shown) may be used. In the case of more complex 3D surfaces, a controlled plasma module (not shown) may move around a stationary target or the surface to be exposed to the plasma may be placed on a movable stage. In some embodiments, one or more plasma jets or can be attached to a robotic arm that is programmed to move in a manner that exposes one or more target areas to a plasma plume or jet.

In addition, in some embodiments, the plasma generator 201 may be coupled with a biomolecule/drug delivery system, where molecules may be transported to the treatment area through needle-free injection, evaporation, spraying and or misting. In some embodiments, this may assist with the pretreatment of the surface.

In some embodiments where it is essential to reduce the plasma temperature and enhance skin permeation following plasmaporation it is beneficial to generate non-thermal plasma using He, Ar, Ne, Xe and the like, air, or mixtures of inert gases with small percentage (0.5%-20%) of other gases such as O₂ and N₂ and mixtures of inert gases with vaporized liquids including water, DMSO, ethanol, isopropyl alcohol, n-butanol, with or without additives and the like.

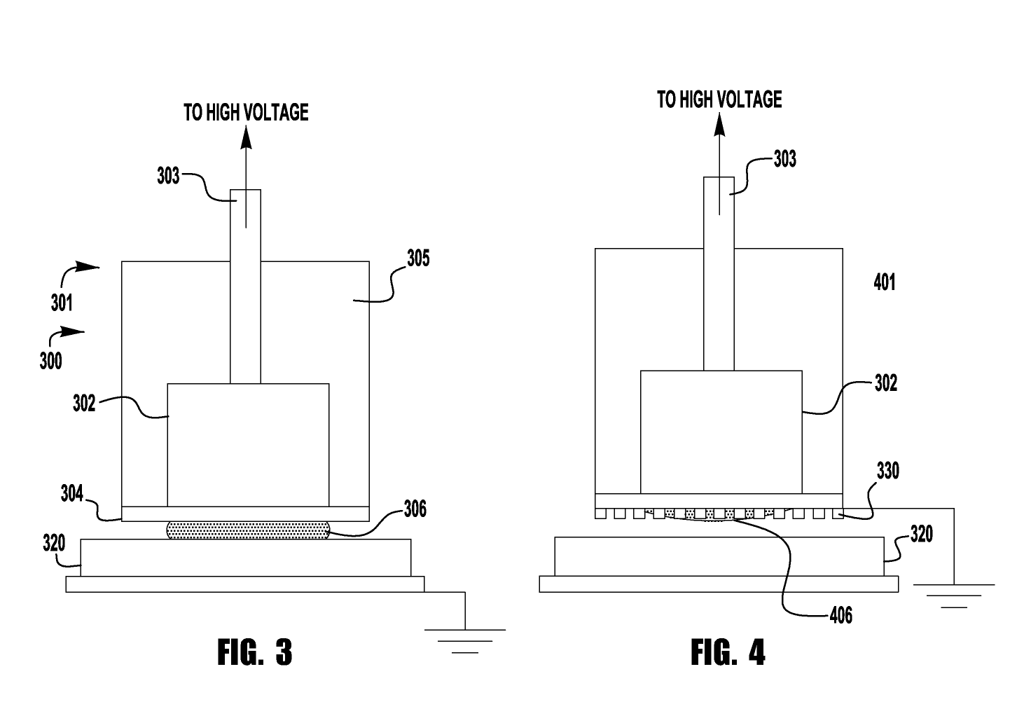

FIG. 3 illustrates another exemplary transdermal delivery system 300. Transdermal delivery system 300 includes a plasma generator 301. Plasma generator 301 includes a high voltage wire 303 connected to an electrode 302 on a first end and a high voltage power supply (not shown) on the second end. Suitable high voltage supplies are described above. In some of the exemplary embodiments, the power supply was a nanosecond pulsed power supply. The plasma 306 was generated by applying alternating polarity pulsed voltage with nanosecond duration pulses. The applied voltage had a pulse width of between about 40-500 ns (single pulse to 20 kHz) with a rise time of 0.5-1 kV/ns and a magnitude of about ˜20 kV (peak-to-peak) at a power density of 0.01-100 W/cm². A dielectric barrier 304 is located below the high voltage electrode 302. In addition, the high voltage electrode 302 is located within a housing 305. Plasma generator 301 is a non-thermal dielectric barrier discharge (DBD) generator. Plasma 306 is generated by the plasma generator 301. FIG. 3 also includes skin 320. The skin or tissue acts as the second electrode, which may be grounded or may be a floating ground. Plasma 306 is in direct contact with the skin 320. For the exemplary experimental results disclosed herein, skin 320 is porcine skin.

Direct plasma 306 was generated by applying alternating polarity pulsed voltage to the electrode 302. The applied voltage had a pulse width between about 1-10 μs (100 Hz to 30 kHz) with a magnitude of about 20 kV (peak-to-peak). The power supply (not shown) was a variable voltage and variable frequency power supply. A 1 mm thick clear quartz slide was used as the insulating dielectric barrier 304 and it covers the electrode 302. Electrode 302 was a 2.54 cm diameter copper electrode. The discharge gap between the dielectric barrier 304 and the porcine skin 320 was about 4 mm±1 mm. In some experiments, the pulse waveform had an amplitude of about 22 kV (peak-to-peak), a duration of about 9 μs, with rise time of about 5 V/ns. The discharge power density was between about 0.1 W/cm² to 2.08 W/cm². The plasma treatment dose in J/cm² was calculated by multiplying the plasma discharge power density by the plasma treatment duration.

In addition, indirect plasma 406 was created with a plasma generator 401. Plasma generator 401 is similar to plasma generator 301, except that plasma generator 401 includes a metal mesh 330 that filters the plasma 406. The metal mesh 300 prevents charged ions and electrons from passing through, but allows the neutral species to pass through and contact the skin. The neutral species may be referred to as “afterglow.”

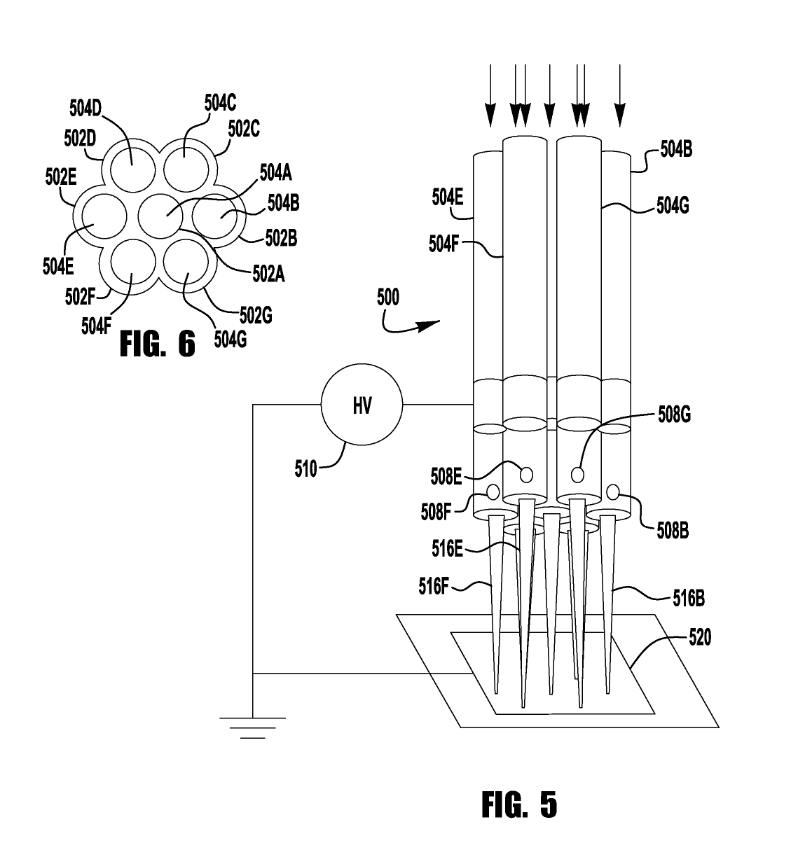

FIG. 5 is a schematic of yet another exemplary embodiment of a transdermal delivery system 500. FIG. 6 is a plan view of the electrodes of transdermal delivery system 500. Transdermal delivery system 500 includes a plurality of DBD jets. The exemplary transdermal delivery system 500 has an array of DBD jets in a honeycomb shape; however, many other configurations may be used such as, linear, triangular, square, pentagonal, hexagonal, octagonal, etc.

The DBD jets have glass tubes 504A, 504B, 504C, 504D, 504E, 504F and 504G. A metal electrode 502 includes a plurality of cylindrical openings 502A, 502B, 502C, 502D, 502E, 502F, and 502G that receive each of the corresponding glass tubes 504A, 504B, 504C, 504D, 504E, 504F, and 504G. Optionally, multiple metal electrodes may be used. The metal electrode 502 may have an insulating covering (not shown) to prevent shock. The metal electrode 502 is connected to a high voltage source as described above.

The DBD jets have a gas flow inlet located at a first end and have a plasma jet 516A, 516B, 516C, 516D, 516E, 516F and 516G out the other. As described above, the gas may be, for example, He, Ar, Ne, Xe, air, He+Air, Ar+Air, Ne+Air, Xe+Air, or the like. In addition, each glass tube 504A, 504B, 504C, 504D, 504E, 504F and 504G has an inlet 508A, 508B, 508C, 508D, 508E, 508F, and 508G located along the glass tube for receiving vaporized liquid additives. These inlets may be located above or below electrode 502. The exemplary transdermal delivery system 500 utilizes skin as a ground electrode.

In the various experiments described herein, some embodiments of transdermal delivery systems used the direct plasma generator 201 described with respect to FIG. 2, some used the direct plasma generator 301 described with respect to FIG. 3, and some used the indirect plasma generator described with respect to FIG. 4.

In the exemplary embodiment of FIGS. 2 and 3, the skin 220 is directly exposed to the plasma 206 containing energetic electrons, neutral and charged species including negative and positive ions. Similarly, with direct plasma generator 301 the electrical discharge occurred between the dielectric barrier 304 and the skin 320, which exposed the skin directly to energetic electrons, neutral reactive species and charged particles including negative and positive ions.

Indirect plasma created by plasma generator 401 utilized a grounded copper mesh (16×16 mesh size with a 0.011″ wire diameter and a 0.052″ opening size) that was placed between the high voltage electrode and the skin, which eliminated charged particles from contacting the exposed surface of the skin.

### Experimental Results

A number of experiments were conducted and the experimental results and procedures were based on plasma treatment of porcine skin to transport dextran molecules tagged with fluorescent dyes, or proteins tagged with fluorescent dyes or nanoparticles tagged with fluorescent dyes through layers of the skin. The transport of various sized dextran molecules demonstrated the viability of using plasma for transdermal delivery of molecules of different sizes, polarities and physicochemical properties.

Porcine skin with intact stratum corneum (SC) from back of the ear and abdomen were used, which included both full thickness (non-fleshed) and split thickness (fleshed) skin. The skin was kept at −80° C. until the day of treatment. On the day of treatment the skin was thawed to room temperature and kept in a humidified box for 1 hour. Prior to plasma application the hair was removed with a hair clipper and the skin was shaved. The skin was washed with soap and pat-dried with paper towels. The skin from back of the ear was cut in to 1″×1″ pieces and the skin from the abdomen was cut in to 2″×2″ pieces. The pieces of skin were kept in a humidified box on wet paper towels to maintain constant humidity.

Lysine fixable fluorescently tagged dextran molecules having molecular weights of 3, 10, 40 and 70 kDa were also used. Dextrans are not able to freely diffuse through the skin on their own and were used as probes to confirm the methods and apparatuses for plasma-induced poration (“plasmaporation”) processes claimed and described herein. In each of the experiments, dextran molecules were reconstituted in deionized water at a concentration of 5 mg/ml.

In some experiments, the porcine skin was treated with non-thermal DBD plasma for periods of time up to about 3 min and the following plasma power source parameters were varied: the frequency (Hz) was varied between about 100 and about 3500 Hz, the pulse duration was varied between about 1 and about 10 μs; the duty cycle was varied between about 1 to about 100%, and the time of treatment ranged from between about 0.5 to about 3 minutes.

In some experiments, 40 μL of the dextran suspension was applied to skin immediately after plasma treatment. In some experiments, the skin was plasma treated for about 1 minute, followed by application of dextran solution, and then the target area was treated with plasma for about another 1 minute. In some experiments, dextran solution was applied to skin and treated with plasma for about 1 minute. In various experiments, the treated skin was allowed to interact with the dextran solution for 15, 30, 45 or 60 minutes in the dark.

After treatment, 5-10 mm punch biopsies were obtained from control samples and plasma treated samples. The biopsies were immediately submerged in 10% neutral buffered formalin and then stored at 4° C. The biopsies were prepared for histological analysis using paraffin embedding followed by Hematoxylin and Eosin (H&E) staining or by cryostat sectioning. 10 μm slices were obtained perpendicular to the surface of the skin and mounted on glass microscope slides. Morphological analysis and depth of permeation analysis was carried out on an EVOS inverted fluorescence enabled microscope (AMG Microscope).

The experimental results demonstrated non-thermal plasma induces poration in intact porcine skin without visible thermal damage to the underlying skin. In addition, there is evidence that dextran molecules of 3 kDa (1 nm hydrodynamic radius) passed through the skin to an average depth of 500 μm. Dextran molecules of 10 kDa (2 nm hydrodynamic radius) traveled to an average depth of 200 μm. These and more detailed results are provided in Table I below.

The second column of Table I indicates the type of plasma source that was used. The third column indicates the molecular weight of the dextran molecules. The fourth through the sixth columns identifies settings on the power supply for the plasma generator for that particular experiment. The seventh column indicates the time (if any) the treated area was exposed to plasma prior to the solution of dextran molecules being applied to the treated area. Similarly, the eighth column indicates the time (if any) the treated area was exposed to plasma after the solution of dextran molecules was applied to the treated area. The ninth column indicates the amount of time the solution was left on the treated area after plasma treatment and the tenth column indicates the average permeation depth of the dextran molecules in the skin.

The exemplary DBD Jet plasma generator illustrated in FIG. 2 using helium gas as an input and is identified as “He DBD Jet.” The DBD plasma generator illustrated in FIG. 3 is identified as “air DBD.”

TABLE I
Depth of permeation of fluorescently tagged dextran molecules
for different plasma configurations and treatment modalities

| | | | | | | | Post | | |
|---|---|---|---|---|---|---|---|---|---|
| | | | | Pulse | Duty | Pre Plasma | Plasma | Hold | |
| | | Dextran | f | Duration | Cycle | Exposure | Exposure | Time | Depth |
| Sr | Plasma | (kDa) | (Hz) | (μs) | (%) | (min) | (min) | (min) | (μm) |
| 1 | He DBD Jet | 3 | 3500 | 1 | 100 | 1 | 1 | 15 | 360 |
| 2 | He DBD Jet | 3 | 3500 | 1 | 100 | NA | 1 | 15 | 550 |
| 3 | He DBD Jet | 3 | 3500 | 1 | 100 | 1 | NA | 15 | 350 |
| 4 | He DBD Jet | 3 | 2000 | 1 | 100 | 1 | 1 | 15 | 250 |
| 5 | He DBD Jet | 3 | 500 | 1 | 100 | 1 | 1 | 15 | 535 |
| 6 | He DBD Jet | 10 | 3500 | 9 | 90 | 1 | NA | 15 | 315 |
| 7 | He DBD Jet | 10 | 3500 | 1 | 100 | 1 | 1 | 15 | 88 |
| 8 | Air DBD | 3 | 3500 | 10 | 80 | NA | 1 | 10 | 133 |
| 9 | Air DBD | 3 | 3500 | 10 | 80 | NA | 1 | 45 | 250 |
| 10 | Air DBD | 3 | 3500 | 10 | 80 | NA | 3 | 45 | 171 |
| 11 | Air DBD | 3 | 3500 | 5 | 100 | 1 | NA | 60 | 220 |
| 12 | Air DBD | 3 | 3500 | 5 | 100 | 3 | NA | 60 | 161 |
| 13 | Air DBD | 3 | 2500 | 10 | 50 | 10 | NA | 60 | 150 |

The average depth of the SC is between about 10 μm and 20 μm. Accordingly, all of the experimental results above demonstrated that plasmaporation was successful in delivering molecules through the SC, which is the main barrier to transdermal delivery.

In addition, decreasing the pulse duration of the He DBD jet resulted in an increase in the depth of permeation in to the skin. Increasing the plasma frequency also increased the depth of permeation into the skin. Increasing the duty cycle increased the depth of permeation in to the skin as well. It was also discovered that short plasma treatment times (on the order of 1 minute) yielded greater depth of permeation in to the skin. In addition, increased depth of permeation for higher molecular weight dextrans was observed at longer pulse durations.

Further, use of a DBD Jet plasma generator for the plasma treatment yielded on average a higher depth of permeation than plasma generators generating plasma using regular DBD. In addition, the depth of permeation was limited to the epidermis when using plasma generated by a plasma generator having regular DBD electrode at longer pulse durations and shorter duty cycles. Accordingly, the results demonstrate that the depth of permeation of the molecule of interest can be controlled by varying plasma treatment parameters which include, for example, the type of plasma generator used, frequency, duty cycle, pulse duration, time of plasma treatment and time of application on the skin.

The application of the molecule of interest before or after plasma treatment yielded similar depths of permeation after similar time of plasma exposure. Thus, application of plasma prior to the application of sensitive molecules, drugs or vaccines allows the permeation of sensitive molecules, drugs or vaccines through newly created pores without degradation or loss of activity of the sensitive molecule drug or vaccines due to interaction with plasma or associated electric fields.

Table II below, identifies a list of exemplary compounds with molecular weights that may be delivered through the skin using plasmaporation. The charge of the compound is also included in the chart.

TABLE II
Molecules Suitable for Transdermal Drug Delivery

| Compound | Molecular Weight/Size | Charge |
|---|---|---|
| Water | 18 | 0 |
| Vitamin C | 176 | 0 |
| Mannitol | 182 | 0 |
| Lidocaine | 234 | 0 |
| Atenolol | 266 | 1 |
| Metoprolol | 267 | 1 |
| Tetracaine | 301 | −1 |
| Alnitidan | 302 | +1/+2 |
| Timolol | 316 | 1 |
| Methylene blue | 320 | 1 |
| Fentanyl | 336 | 1 |
| Na nonivamide | 350 | −1 |
| Nalbuphine | 357 | 1 |
| FITC 390 | 390 | −1 |
| Domperidone | 426 | 1 |
| Lucifer yellow | 457 | −2 |
| Terazosin | 460 | 0 |
| Buprenorphine | 504 | 1 |
| Sulforhodamine | 607 | −1 |
| Calcein | 623 | −4 |
| Erythrosin derivative | 1025 | −1 |
| Cyclosporine A | 1201 | 0 |
| Salmon calcitonin | 3600 | 1 |
| Dextran sulfate | 5000 | Highly negative |
| Heparin | 12000 | Highly negative |
| Defibrase | 36000 | Highly negative |
| Ovalalbumin | 45000 | 0 |
| Antigens | 8-10000 | 0 |
| FITC - dextran | 4-38,000 | negative |
| Nano-microspheres | 10 nm-45 um | highly negative |
| DNA (plasmid) | 20-250 nm | negative |

Although some of the molecules listed in the table above have a MW of less than 500 Dalton and may be suitable for transdermal delivery without plasmaporation, plasmaporation may increase the speed and efficiency of delivery of a therapeutic amount of the molecules, or reduce the need for messy gels or creams.

In addition, plasmaporation may be used to deliver albumin through the SC and into the epidermis. Experimental results demonstrated that albumin (66 kDa) tagged with green fluorescence (a fluorescence tag enables detection of the molecule of interest through standard fluorescent enabled imaging techniques) could be delivered transdermally by treating the skin with plasma. In some experiments, a power source set at 200 ns and 20 kV was used with various pulses. Three (3) pulses, 5 pulses and 10 pulses were applied to the skin and all resulted in permeation of the SC, with 10 pulses delivering the albumin deeper than 3 pulses. Another set of experiments treated the skin with power settings at 200 ns and 5 pulses with different voltages. Ten (10) kV, 15 kV and 20 kV were used. The albumin permeated through the skin with all of the voltages, with 20 kV resulting in the deepest permeation. In addition, treatment using a power source set at 100 ns and 30 second with several different frequency settings resulted in different permeation depths. 500 Hz, 1000 Hz and 5000 Hz all result in permeation into the epidermis, with the high frequency resulting in the deepest permeation. Experimental results indicate that albumin was predominately localized in the epidermis up to a depth of between about 75 and 100 μm.

In addition, plasmaporation may be used to deliver fluorescently tagged IgG (human immunoglobulin G) through the SC and into the epidermis and dermis. Experimental results demonstrated that IgG (115 kDa) could be delivered transdermally by treating the skin with microsecond pulsed plasma. In some experiments, a power source set at 200 ns and 5 μs was used to treat skin for 30 seconds with various frequencies and the IgG was applied to the skin for a 60 minutes hold time. 500 Hz, 1500 Hz and 3500 Hz all result in permeation into the epidermis, with the high frequency resulting in the deepest permeation. In some experiments, a power source set at 200 ns and 3500 Hz was used to treat skin for 30 seconds with various pulse durations and the IgG applied to the skin for a 60 minutes hold time. 1 μs, 3 μs, and 5 μs pulses were used and all result in permeation into the epidermis, with the high frequency resulting in the deepest permeation. Accordingly, the depth of delivery of IgG via microsecond pulsed plasma induce portion is proportional to the frequency of plasma in the PD (application of plasma to skin followed by application of the molecule) mode, while in the PDP (application of plasma to skin followed by application of the molecule and then a second application of plasma to skin) mode it is strongly dependent on the pulse duration. PDP mode enhances permeation of IgG deeper into the skin than PD mode. Experimental results indicate that IgG was predominately localized in the epidermis, but strong signals were determined in the dermis at between about 400 and 600 μm.

Table III identifies a list topical drugs that are currently applied to the skin. These topical drugs may be applied in a gel or cream. In some exemplary methodologies, these drugs may be applied after plasmaporation, without the need for the messy gels or creams. In addition, plasma poration may reduce the amount of time required to deliver a therapeutic amount of the drug. Moreover, applying the compounds after plasma treatment allows the topical drugs to rapidly penetrate the SC without altering the composition. Because the methods disclosed herein do not alter the chemical composition, obtaining FDA approval for a new drug or composition may not be needed, or the speed of approval may be increased. In addition, the topical drugs may be applied without the gel or cream (or with less gel or cream.) In addition, less of the drug may be required because the absorption rate is increased.

TABLE III
Commonly Used Topical Drugs

| Compound | Molecular weight (Da) |
|---|---|
| Topical antifungals: | |
| Ketoconazole | 531 |
| Clotrimazole | 345 |
| Terbinafine | 291 |
| Miconazole | 416 |
| Topical corticosteroids | |
| Hydrocortisone acetate | 404.5 |
| Bethamethasone valerate | 477 |
| Diflucortolone valerate | 394 |
| Clobetasol propionate | 467 |
| Mometasone fuorate | 521 |
| Topical anti-infectives | |
| Fucidic acid | 517 |
| Gentamycin | 478 |
| Acyclovir | 225 |

Table IV identifies a list of drugs that are currently used in transdermal drug-delivery systems that may be administered using plasmaporation in less time, or without the need for messy creams and gels. The benefits described above with respect to Table III also apply to the List in Table IV.

TABLE IV
Transdermal Drugs

| Compound | Molecular weight (Da) |
|---|---|
| Scopolamine | 305 |
| Nitroglycerine | 227 |
| Nicotine | 162 |
| Clonidine | 230 |
| Fentanyl | 336 |
| Oestradiol | 272 |
| Testosterone | 288 |

Plasmaporation has a number of other practical applications. In some embodiments, plasmaporation may be used to increase permeation of sanitizers, antimicrobials, surgical scrubs, and the like. Exemplary sanitizers, antimicrobials, surgical scrubs are identified in Table V below.

TABLE V
Sanitizers and Antimicrobials

| Compound | MW (Da) |
|---|---|
| Chlorhexidine gluconate | 700 |
| Neomycin | 615 |
| Chlorhexidine | 505 |
| Povidone iodine | 364 |
| Hydrocortisone | 362 |
| Triclosan | 289 |
| Chloroxylenol | 156 |

Increasing the permeation of antimicrobials, for example, increases the efficacy and rate of kill of undesirable microbes in deeper layers of skin. In addition, certain antimicrobials take a long time to penetrate cell walls; however, plasmaporation increases the permeation rate and accelerates the kill time.

Plasmaporation may be also be used to treat acne. Plasmaporation may open the existing clogged pores as well as surrounding pores and sterilize the infected area. Second, plasmaporation allows antimicrobials and other acne medication to enter the pores. Thus, rather than take medications that have serious side effects, plasmaporation may be used without the side effects. In addition, the plasma treatment may not need to be used on a daily basis and may be used at predetermined intervals, such as once a week, a few times a week or the like to treat acne. In some embodiments, the plasma treatment is only needed periodically.

Plasmaporation may be also used to open pores and drive cosmetic related materials, such as, for example, collagen, BOTOX or other fillers into the skin to reduce wrinkles Table VI identifies exemplary cosmetics suitable for use with plasmaporation.

TABLE VI
Skin Fillers

| Compound | MW | |
|---|---|---|
| Collagen | 120-250 | kDa |
| BOTOX | 150 | kDa |

| | |
|---|---|
| Hyaluronic acid | 5000 Da-20 MDa (typically |
| | used at 3000-8000 Da) |

Plasmaporation may be used to increase the absorption rate of moisturizers and thereby minimizes the “tack” associated with moisturizers that have not been fully absorbed. Heavy moisturizes that would not ordinarily penetrate into the skin do so after plasmaporation. Exemplary heavy moisturizers suitable for use with plasmaporation are identified in Table VII below.

TABLE VII
Heavy Moisturizers

| Compound | MW | |
|---|---|---|
| Dimethicone | 50-1000 | Da |
| Hyaluronic acid | 3000-8000 | Da |
| Polyethylene glycol stearate | 100-1000 | Da |
| Petrolatum | 350-650 | Da |

| | |
|---|---|
| Oleic Acid | 282 |
| Glycerin | 92 |

In some embodiments, the skin may be preconditioned to temporarily alter the skin pH, moisture level, temperature, electrolyte concentration or the like. Preconditioning helps maximize speed and depth of permeation of active ingredients through pore formation without harming the skin.

In some embodiments, plasmaporation alone or in combination with hand-washing solutions may be used to achieve permeation of surface active agents to superficial depths of the skin, which enables more effective release and removal of soils embedded that are adhered to the skin. Exemplary surface-active agents are produced below in Table VIII.

TABLE VIII
Harsh surface-active Agents

| | | MW |
|---|---|---|
| Class | Exemplary Compounds | (Da) |
| Chlorhexidine and other | Chlorhexidine gluconate | 700 |
| diguanides | | |
| Iodine based compounds | Providone Iodine (Betadine) | 364 |
| Alcohols | Ethyl Alcohol (70%) | 46 |
| Peroxides and permanganates | Hydrogen peroxide solution | 34 |

In addition, plasmaporation may enable use of less chemically aggressive surface-active agents and/or lower concentrations and volumes. Exemplary less harsh surface-active compounds are shown in Table IX below.

TABLE IX
Less Harsh Surface-Active Compounds

| | | MW |
|---|---|---|
| Class | Exemplary Compounds | (Da) |
| Quaternary Ammonium | Benzalkonium chloride | 360 |
| compounds | | |
| Antibacterial dyes | Proflavine hemisulphate | 307 |
| Halogenated phenol derivatives | Chloroxylenol | 156 |
| Quinolone derivatives | Potassium hydroxyquinoline | 281 |
| | sulphate | |

In some embodiments, plasmaporation may be used in combination with low levels of non-irritating chemical skin permeation enhancers to achieve synergistic permeation of actives, including antimicrobials, cosmetic ingredients, vaccines, or drugs. Examples of chemical enhancers include dimethyl sulfoxide, azone, pyrrolidones, oxazolidinones, urea, oleic acid, ethanol, liposomes. Molecular weights of exemplary chemical permeation enhancers are shown below in Table X.

TABLE X
Skin Permeation Enhancers

| Compound | MW |
|---|---|
| Oleic Acid | 282 |
| Azone (Laurocapram) | 281 |
| Lauric Acid | 200 |
| Oxazolidinones | 90 |
| Pyrrolidones | 85 |
| DMSO | 78 |
| Ethanol | 46 |

As described above, plasmaporation may be used to drive common topical drugs into the skin faster. Advantages of delivering common topical drugs into the skin faster include, maintaining tighter therapeutic concentrations, eliminating the need for mixing the topical drugs with other compounds such as messy gels for proper absorption. The methodology may result in no need for additional FDA approval or increased speed of approval.

In addition, in some embodiments, antioxidants that are designed to achieve an optimal balance of skin permeation performance and skin safety are delivered during plasmaporation to neutralize the oxidizers contained in the plasma to avoid an over-dosing that may cause an adverse immune response or mutagenic damage to DNA in cells within the viable epidermis.

Combination of non-thermal plasma gas, electric field and chemical oxidizers with controlled programming of adjustable variables over time can achieve optimal results for treatment of skin or biofilms.

Although many of the exemplary methods above relate to molecules, particles having similar molecular weights or equivalent diameters may also be transported across layers of the skin. In some embodiments, nanoparticles, such as, for example, silver nanoparticles, silver ions and other metal or polymer nanoparticle are driven into pores in the skin where they are allowed to react. Silver, copper and other metals are known to induce cell lysis and inhibit cell transduction. The introduction of silver and other metals in the form of nanoparticles increases the surface area available to react with microorganisms and enhances the antimicrobial action. Additionally, introduction of nanoparticles that encapsulate the molecule, vaccine, or drug of interest after plasmaporation allows permeation of such molecules to a controlled depth leading to controlled long term release of actives within a particular area of skin. Nanoparticles, including quantum dots, nanotubes and the like, having a diameter of between about 2 and about 400 nanometers may be driven across the skin using plasmaporation.

In additional experiments, it has been discovered that the depth of permeation of a 3 kDa, dextran molecule is directly proportional to the duty cycle of the applied voltage.

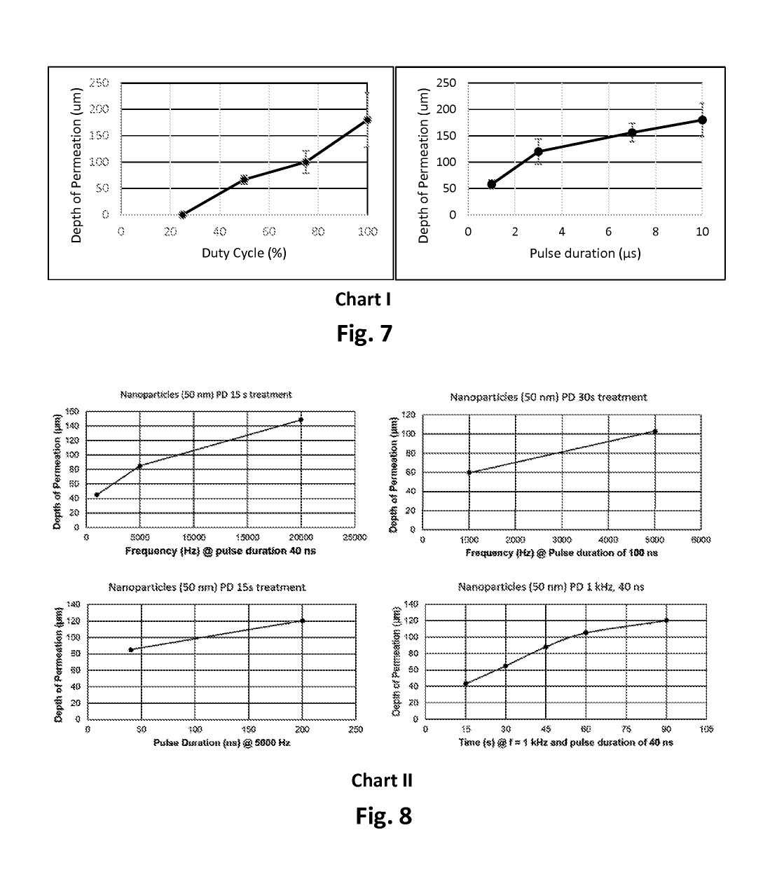

The experiment was conducted using a helium DBD plasma jet with a 5 mm gap between the plasma jet and the surface of the skin. The skin was treated with plasma for 30 seconds at 3500 Hz with a microsecond pulsed power supply. As can be seen in Chart I (FIG. 7), increasing the pulse duration of the applied voltage results in an increase of the depth of permeation of dextran molecules.

Another experiment demonstrated the permeation of nanoparticle after plasma treatment. Ex vivo pig-skin was treated with plasma using a nanosecond pulsed power supply. 100 μl of 50 nanometer fluorescently tagged silica nanoparticles in an aqueous solution were applied to the treated area for between 15 minutes and 1 hour. Biopsies were taken to obtain cryostat processed slides that were imaged. It was discovered that at a fixed pulse duration and a fixed time of treatment, the depth of permeation increased as the frequency of plasma increased. At a fixed frequency and fixed time of treatment the depth of permeation increases as the pulse duration of the plasma increases. At a fixed frequency and fixed pulse duration of plasma, the depth of permeation increases as the time of treatment increases. It was also discovered that 15 seconds of plasma treatment will drive nanoparticles to a depth of about 175 μm and a 30 second treatment at 1 kHz can drive nanoparticles to a depth of about 222 μm. These results are shown below. As can be seen in Chart II (FIG. 8) an increase the number of applied pulses leads to an increase of the permeation depth. Increase of the volume of applied nanoparticles results initially in increase of the permeation depth and then saturates. Similarly, by increasing the concentration of the applied nanoparticles, an increase of the depth of permeation up to a certain depth is observed, and then saturates. The depth of permeation is directly dependent to the applied pulse duration and the frequency of the pulse application. In addition, it was discovered that application of discrete nanosecond duration pulses achieved similar or better results than continuous application. It was also discovered that decreasing the pulse duration, increasing the frequency or increasing the duty cycle increases the depth of permeation into the skin. Longer pulse durations resulted in shallower depth of permeation. Surprisingly, shorter treatment times yielded greater depth of permeation into the skin.

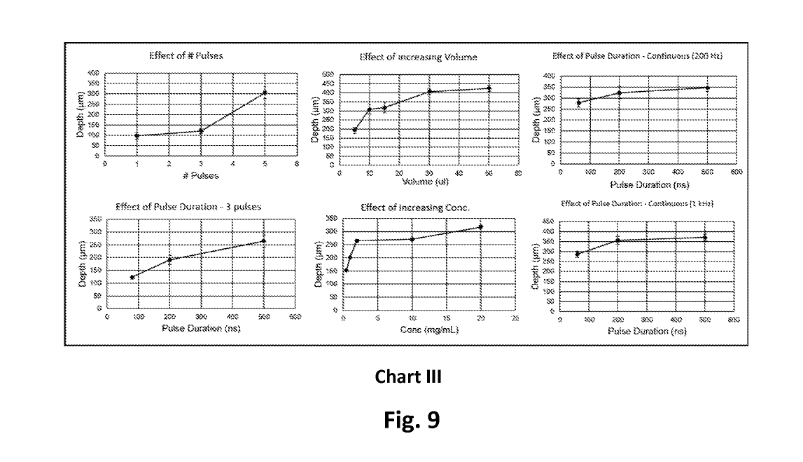

Similar to Chart II (FIG. 8), as can be seen in Chart III (FIG. 9) an increase the number of applied pulses leads to an increase of the permeation depth of applied molecules. Increase of the volume of applied molecules results initially in increase of the permeation depth and then saturates. Similarly, by increasing the concentration of the applied molecule, an increase of the depth of permeation up to a certain depth is observed, and then saturates. The depth of permeation is directly dependent to the applied pulse duration and the frequency of the pulse application. In addition, it was discovered that application of discrete nanosecond duration pulses achieved similar or better results than continuous application. It was also discovered that decreasing the pulse duration or increasing the frequency and/or increasing the duty cycle increases the depth of permeation into the skin. Longer pulse durations resulted in shallower depth of permeation. Surprisingly, shorter treatment times yielded greater depth of permeation into the skin.

While the exemplary embodiments are illustrated using skin, any of the described embodiments would work equally well with any tissue including epithelial tissue; mucosal epithelial tissue; muscle tissue, connective tissue; inner and outer lining of organs.

While the present invention has been illustrated by the description of embodiments thereof and while the embodiments have been described in considerable detail, it is not the intention of the applicant to restrict or in any way limit the scope of the appended claims to such detail. Additional advantages and modifications will readily appear to those skilled in the art. Therefore, the invention, in its broader aspects, is not limited to the specific details, the representative apparatus and illustrative examples shown and described. Accordingly, departures may be made from such details without departing from the spirit or scope of the applicant's general inventive concept.

We claim:

1. A method of delivering or moving a molecule into skin comprising:
opening one or more pores in skin by applying a plasma field to skin;
followed by the application of a carrier, selected from one of a cream, a patch, a gel, an ointment, an aerosol, or a liquid and having one or more molecules of molecular weight greater than 500 Da on the surface of the skin;
allowing one or more molecules to pass through the pores to a desired depth; and
allowing the one or more pores to close.

2. The method of claim 1 wherein the carrier comprises molecules dissolved in a solution.

3. The method of claim 1 wherein the carrier comprises molecules in a suspension.

4. The method of claim 1 wherein the carrier comprises charged molecules.

5. The method of claim 1 wherein the one or more molecules have a molecular weight of greater than 1000 Da.

6. The method of claim 1 wherein the one or more molecules have a molecular weight of greater than 3000 Da.

7. The method of claim 1 wherein the one or more molecules have a molecular weight of greater than 10000 Da.

8. The method of claim 1 wherein the one or more molecules comprise moisturizing molecules.

9. The method of claim 1 wherein the one or more molecules comprise cosmetic molecules.

10. The method of claim 9 wherein the cosmetic molecules comprise a filler.

11. The method of claim 10 wherein the filler comprises collagen.

12. The method of claim 1 wherein the one or more molecules comprise sanitizing molecules.

13. The method of claim 1 wherein plasma is applied to the surface of the skin, the carrier including one or more molecules is applied on the surface of the skin for a predetermined amount of time and then plasma is applied to the surface of the skin after the one or more molecules pass through the pores created by the first plasma application.

14. The method of claim 13 wherein the time between the application of the carrier containing one or more molecules and the second plasma treatment is between about 1 seconds and about 120 seconds.

15. The method of claim 1 wherein the plasma field is generated by a plasma generator that generates continuous plasma in contact with the skin for between about 1 second and about 120 seconds.

16. The method of claim 1 wherein the plasma field is generated by a plasma generator set with a pulse repetition frequency of between about 2 and 20000 Hz.

17. The method of claim 1 further comprising setting the pulse duration of a plasma generator for generating the plasma to between about 1 μs and about 10 μs.

18. The method of claim 1 further comprising setting the pulse duration of a plasma generator for generating the plasma to between about 0.1 nanosecond and about 500 nanosecond.

19. The method of claim 1 further comprising setting the duty cycle of a plasma generator for generating the plasma to between about 10 and about 100%.

20. The method of claim 1 wherein the plasma is generated by a plasma generator and the plasma generator is a dielectric barrier discharge jet plasma generator.

21. The method of claim 1 wherein the plasma is generated by a plasma generator and the plasma generator is a dielectric barrier discharge jet plasma generator and has a helium gas feed.

22. The method of claim 1 wherein the plasma is generated by a plasma generator and the plasma generator is a dielectric barrier discharge plasma generator using ambient air to create plasma.

23. The method of claim 1 wherein the plasma is generated by a plasma generator and the plasma generator has a microsecond pulsed power supply.

24. The method of claim 1 wherein the plasma is generated by a plasma generator and the plasma generator has a nanosecond pulsed power supply.

25. The method of claim 24 wherein the nanosecond pulsed power supply applies plasma to skin in a number of discrete pulses

26. The method of claim 24 where the number of discreet pulses could range from between about 1 to about 100 discrete pulses having a pulse duration between about 1 ns and about 500 ns

27. The method of claim 1 wherein the one or more molecules are driven to an average depth of between about 30 and 600 μm.

28. The method of claim 1 wherein the molecule one or more molecules are driven to an average depth of between about 125 and 500 μm.

29. The method of claim 1 wherein the molecule one or more molecules are driven to an average depth of between about 200 and 400 μm.

30. The method of claim 1 wherein the one or more molecules are driven to an average depth of between about 200 and 1000 μm.

31. The method of any of claim 1 further comprising preconditioning the skin prior to applying plasma to the skin.

32. The method of claim 31 wherein the preconditioning consists of altering at least one of skin pH, moisture level, temperature or electrolyte concentrations.

33. The method of claim 31 further comprising applying a chemical skin permeation enhancer to the skin.

34. The method of claim 33 wherein the chemical skin permeation enhancer is one of dimethyl sulfoxide, oleic acid, or ethanol.

35. A method of delivering or moving nanoparticles into skin comprising:
opening one or more pores in skin by applying a plasma field to skin;
applying one or more nanoparticles having a size of less than 600 nm on the surface of the skin;
allowing the one or more pores to transport one or more nanoparticles through the one or more pores; and
allowing the one or more nanoparticles to pass through the one or more pores to a desired depth; and
allowing the one or more pores to close.

36. The method of claim 35 wherein the one or more nanoparticles comprise silver, zinc oxide, titanium dioxide, silica, chitosan or quantum dots.

37. The method of claim 35 wherein the one or more nanoparticles encapsulate one or more molecules, vaccines, or drugs.

38. A method of increasing the speed of permeation of transdermal drugs or topical drugs comprising:
opening one or more pores in skin by applying a plasma field to skin;
applying a topical drug or transdermal drug on the skin; and
allowing the transdermal drug or topical drug to pass through the one or more pores to a desired depth.
and allowing the one of more pores to close.
